(19) United States
(12) Patent Application Publication
(10) Pub. No.: US 2019/0239511 A1
(43) Pub. Date: Aug. 8, 2019

(54) Synergistic Combinations Of Monochloramine And Peroxide Compound, And Methods Of Using The Same For Microbial Control

(71) Applicant: Buckman Laboratories International, Inc., Memphis, TN (US)

(72) Inventors: John P. Buyondo, Arlington, TN (US); Mark L. Reed, Cordova, TN (US); Bernard Janse, Collierville, TN (US)

(73) Assignee: Buckman Laboratories International, Inc., Memphis, TN (US)

(21) Appl. No.: 16/242,021

(22) Filed: Jan. 8, 2019

Related U.S. Application Data
(60) Provisional application No. 62/627,210, filed on Feb. 7, 2018.

(51) Int. Cl. A01N 59/00; A01N 25/04

(52) U.S. Cl. CPC A01N 59/00; C12P 7/06; A01N 25/04

(57) ABSTRACT

Methods for controlling the growth of microorganisms in or on a product, material, or medium, such as a fermentable or fermenting medium, susceptible to attack by a microorganism, by treating with aqueous solution comprising monochloramine and at least one peroxide compound in a synergistically microbicidally effective combined amount to control unwanted microbial growth. Microbicidal aqueous solutions containing monochloramine and at least one peroxide in a synergistically microbicidally effective combined amount to control the growth of at least one microorganism are also described.

### BACKGROUND OF THE INVENTION

This application claims the benefit under 35 U.S.C. § 119(e) of prior U.S. Provisional Patent Application No. 62/627,210, filed Feb. 7, 2018, which is incorporated in its entirety by reference herein.

The present invention relates to synergistic combinations of antimicrobials in aqueous solutions or formulations and methods of their use for controlling the growth of microorganisms on a variety of mediums, substrates, and in liquid systems, such as ethanol fermentation systems. More particularly, the present invention relates to using monochloramine and peroxide compound, such as hydrogen peroxide, in aqueous treatment solutions and/or for treatment of aqueous systems.

Many industrial materials and media when wet or subjected to treatment in water are susceptible to bacterial, fungal, and/or algal deterioration or degradation. A large variety of commercial, industrial, agricultural, and wood materials or products are subject to microbiological attack or degradation which reduces or destroys their economic value. These industrial materials and media include, but are not limited to, for example, wood pulp, wood chips, lumber, adhesives, coatings, animal hides, paper mill liquors, pharmaceutical formulations, cosmetic formulations, toiletry formulations, geological drilling lubricants, petrochemicals, agrochemical compositions, paints, leathers, plastics, seeds, plants, wood, metalworking fluids, cooling water, recreational water, influent plant water, waste water, pasteurizers, retort cookers, tanning liquors or solutions, starch, proteinaceous materials, acrylic latex paint emulsions, and textiles. The various temperatures at which such materials or products are manufactured, stored, or used as well as their intrinsic characteristics make them susceptible to growth, attack, and degradation by common microorganisms such as algae, fungi, yeasts, and bacteria. These microorganisms may be introduced during a manufacturing or other industrial process, by exposure to air, tanks, pipes, equipment, and humans. They can also be introduced while using a material or product, for example, by multiple openings and reclosures of packages or from stirring or removing material with contaminated objects.

To control deterioration or degradation caused by microorganisms, various industrial microbicides are used. Workers in the trade have continued to seek improved biocides that have low toxicity, are cost effective, and/or are also capable of exhibiting a prolonged biocidal effect against a wide variety of microorganisms with regular use.

Aqueous systems are also highly subject to microbiological growth, attack, and degradation. These aqueous systems may be fresh, brackish or saltwater systems. Exemplary aqueous systems include, but are not limited to, latexes, surfactants, dispersants, stabilizers, thickeners, adhesives, starches, waxes, proteins, emulsifying agents, cellulose products, metal working fluids, cooling water, waste water, aqueous emulsions, aqueous detergents, coating compositions, paint compositions, and resins formulated in aqueous solutions, emulsions or suspensions. These systems frequently contain relatively large amounts of water and organic material causing them to be environments well-suited for microbiological growth and thus attack and degradation.

Microbiological degradation of aqueous systems may manifest itself as a variety of problems, such as loss of viscosity, gas formation, objectionable odors, decreased pH, emulsion breaking, color change, and/or gelling. Additionally, microbiological deterioration of aqueous systems can cause fouling of the related water-handling system, which may include cooling towers, pumps, heat exchangers, pipelines, heating systems, scrubbing systems, and other similar systems.

Another objectionable phenomenon occurring in aqueous systems, particularly in aqueous industrial process fluids, is slime formation. Slime formation can occur in fresh, brackish or salt water systems. Slime consists of matted deposits of microorganisms, fibers and debris. It may be stringy, pasty, rubbery, tapioca-like, or hard, and may have a characteristic undesirable odor that is different from that of the aqueous system in which it formed. The microorganisms involved in its formation are primarily different species of spore-forming and nonspore-forming bacteria, particularly capsulated forms of bacteria which secrete gelatinous substances that envelop or encase the cells. Slime microorganisms also include filamentous bacteria, filamentous fungi of the mold type, yeast, and/or yeast-like organisms. Slime reduces yields in production and causes plugging, bulking, and other problems in industrial water systems.

Some industrial production processes involving fermentation, such as ethanol production processes, need microbial growth control. In these process environments, it is desirable to control unwanted microbes that can contaminate these processes without harming beneficial microbes present or used in the system.

In ethanol production, ethanol can be produced by fermentation using a wide variety of starch containing raw materials. Starch-based ethanol production generally includes preparing a mass of starchy feedstock that contains or can be degraded into fermentable sugars, adding water to make a mash, enzymatic liquefaction/saccharification of carbohydrates into fermentable sugars, and adding yeast which ferments the sugar into ethanol and carbon dioxide. Ethanol is recovered by subjecting the fermented mash to distillation. A co-product of distillation in ethanol production is non-starchy solids containing proteins, fibers, and oils, which may be processed to produce “distillers dried grains with solubles” or “DDGS”. DDGS are nutrient-rich and are commercially sold as an animal feed, feed supplement, or plant fertilizer.

A problem in the ethanol production industry is that the ethanol fermentation system can become contaminated with bacteria that reduce production yields. This contamination can occur in one or more vessels used in holding, propagation and fermentation, including pre-fermentation holding tanks, propagation tanks, fermentations tanks, and piping and process equipment between these units. “Lactic acid bacteria” is one class of bacteria that poses a problem in this respect. Lactic acid bacteria include, for example, Lactobacillus, Pediococcus, Leuconostoc and Weissella species. Acetic acid bacteria, e.g., Acetobacter sp., can also cause problems by producing acetic acid, lactic acid, or other organic acids which foul the process and reduce the yields of ethanol. Yeast converts sugars to ethanol, but bacteria also convert those same sugars to make lactic or acetic acid instead of ethanol, leading to reductions in ethanol production yield. To control the outbreak of such bacteria, antibiotics, for example, virginiamycin, penicillin, erythromycin, and tylosin, have been used in ethanol fermentation processes. The risk of the bacteria developing drug-resistance to antibiotics from their use or overuse is a concern. Further, questions have been raised about non-specificity of the antibiotic to the target bacteria and fermentation products. Concerns also have been raised about the presence of antibiotic residues in the DDGS destined for animal feeds. Alternatives to antibiotics are needed for ethanol fermentation processes.

Oxidizing based chemistries proposed for fermentation systems that are based on usage of a single type of microbiocide do not significantly reduce and/or control bacteria growth, or would require significantly high concentration of a microbiocide to control bacterial growth, or are non-selective in anti-microbial action. Chlorine dioxide (i.e., ClO₂), for example, has been proposed as an oxidizing biocide. However, chlorine dioxide is a strong oxidizing agent which has nonselective antimicrobial action. Chlorine dioxide attacks both unwanted bacteria and yeast crucial to the fermentation process. Loss of yeast translates into loss of ethanol yield and/or a “sluggish” fermentation and/or a “stuck” fermentation. Chloride dioxide also generates chloride ions, which can corrode equipment and lead to iron deposits or pitting in the process equipment, as well as release iron and chromium into the process system, which can require costly repairs.

Despite the existence of microbicides, the industry is constantly seeking more cost-effective technology which offers equal or better protection at lower cost and/or lower concentration. The concentration of conventional microbicides and the corresponding treatment costs for such use, can be relatively high. Important factors in the search for cost-effective microbicides include the duration of microbicidal effect, the ease of use, the effectiveness of the microbicide per unit weight, and the ability to displace antibiotics for bacterial control with minimal adverse system or environmental impact of their own.

### SUMMARY OF THE INVENTION

It is a feature of this invention to provide a combination of microbiocides in an aqueous solution capable of synergistically controlling the growth of at least one microorganism, for example, fungi, bacteria, algae, or mixtures thereof, for example, over short or over prolonged periods of time. Methods of controlling the growth of at least one microorganism in or on a product, material, or medium with or in an aqueous solution containing the combination of microbiocides are also features of this invention.

Methods and aqueous solutions for preventing damage during storage or loss of yield in an industrial process caused by undesirable microorganisms, such as undesirable bacteria, fungi, algae, or mixtures thereof, are described.

The present invention, in part, relates to a method of controlling the growth of at least one microorganism in or on a product, material, or medium susceptible to attack by a microorganism. The method includes the step of treating the product, material or medium with aqueous solution comprising (a) monochloramine and (b) at least one peroxide compound, wherein components (a) and (b) are present in a synergistically microbicidally effective combined amount to control the growth of at least one microorganism.

The present invention further provides a method to control growth of at least one contaminant microorganism in a fermentable carbohydrate-containing feedstock. This method includes the step of contacting the fermentable carbohydrate-containing feedstock with (a) monochloramine and (b) at least one peroxide compound, wherein components (a) and (b) are present in a synergistically microbicidally effective combined amount to control the growth of at least one contaminant microorganism in the fermentable carbohydrate-containing feedstock. The present invention, in addition, provides a method for producing ethanol by fermentation with controlled growth of contaminant microorganisms. This method includes the steps of a) adding (a) monochloramine and (b) at least one peroxide compound to fermentable carbohydrate-containing feedstock to provide treated feedstock, wherein components (a) and (b) are present in a synergistically microbicidally effective combined amount to control the growth of at least one contaminant microorganism in the treated feedstock, b) fermenting the treated feedstock in the presence of yeast in a vessel to produce fermented mash comprising ethanol and a solids content, and c) distilling the fermented mash to separate at least a portion of the ethanol from stillage comprising the solids content.

The present invention also provides an aqueous solution or formulation comprising a) monochloramine and b) at least one peroxide, where components a) and b) are present in a combined amount synergistically effective to control the growth of at least one microorganism.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

It is understood that both the foregoing general description and the following detailed description are exemplary and explanatory only, and are not restrictive of the present invention as claimed.

The accompanying drawings, which are incorporated in and constitute a part of this application, illustrate some of the features of the present invention and together with the description, serve to explain the principles of the present invention.

### BRIEF DESCRIPTION OF THE DRAWINGS

FIG. 1 illustrates a process flow diagram of a method of treating an ethanol fermentation system with a combination of monochloramine and peroxide compound to provide synergistic microbicidal control according to an embodiment of the present invention.

FIG. 2 is a bar graph depicting bacterial growth control in corn slurry solution (35 wt %) treated with hydrogen peroxide (100 ppm) and monochloramine added in different concentrations (25 ppm or 50 ppm) at different times, wherein times between peroxide and monochloramine addition are 0, 10, 40 and 60 minutes, or sequential monochloramine addition at both 0 and 40 minutes is used, according to embodiments of the present invention, and a “blank” sample that received no treatment was included for comparison.

FIG. 3 is a bar graph depicting the effect of hydrogen peroxide concentration (100 ppm and 200 ppm) on bacteria growth control in corn slurry solution (35 wt %) treated with hydrogen peroxide and monochloramine (“oxamine”) at different concentrations (25 ppm or 50 ppm), according to embodiments of the present invention, and samples treated with oxamine alone (100 ppm and 200 ppm) for comparison.

FIG. 4 is a bar graph depicting the effect of hydrogen peroxide concentration (100 ppm, 200 ppm and 1000 ppm) on bacteria growth control in corn slurry solution (35 wt %) treated with hydrogen peroxide and monochloramine (“oxamine”) at different concentrations (100 ppm or 200 ppm), according to embodiments of the present invention, and samples treated with oxamine alone (100 ppm and 200 ppm) for comparison.

FIG. 5 is a bar graph depicting the effect, as bacteria reduction in logs, of hydrogen peroxide concentration (100 ppm and 200 ppm) on bacteria growth control in corn slurry solution (35 wt %) treated with hydrogen peroxide and monochloramine (“oxamine”) at different concentrations (25 ppm, 50 ppm, 75 ppm, and 100 ppm), according to embodiments of the present invention, and samples treated with hydrogen peroxide alone (100 ppm or 200 ppm) or oxamine alone (25 ppm, 50 ppm, 75 ppm, or 100 ppm) for comparison.

### DETAILED DESCRIPTION

The present invention provides a method to control the growth of one or more microorganisms in or on a product, material, or medium susceptible to attack or contamination by a microorganism by treatment with an aqueous solution comprising a combination or mixture (or a formulation) of a) monochloramine and b) at least one peroxide compound, such as hydrogen peroxide or other peroxide. The monochloramine and peroxide compound can be preferably present in a combined amount synergistically effective to control the growth of at least one microorganism. Synergistic combinations of these microbiocides used in methods and formulations of the present invention can deliver an antimicrobial effect greater than the sum of the individual microbiocides, and thus can provide an improved performance as compared to combinations which are merely additive in terms of antimicrobial efficiency. The microbicidally or synergistically effective amount can vary in accordance with the material or medium to be treated and can, for a particular application, be routinely determined by one skilled in the art in view of this disclosure. The combined use of a) monochloramine and b) at least one peroxide compound can provide superior microbicidal activity at low concentrations or other concentrations against a wide range of microorganisms. The terms “microbiocide” or “biocide” as used herein, can refer to a chemical substance capable of controlling bacteria in a selective way.

The present invention can be used to provide growth control of at least one contaminant microorganism in any environment where monochloramine is used. The present invention can be used to control microbial growth in higher organic load environments, such as where fermentable carbohydrate-containing feedstock are present in industrial ethanol fermentation processes, pharmaceutical processes, or other fermentation-involved processes.

These methods can include a step of contacting fermentable carbohydrate-containing feedstock with (a) monochloramine and (b) at least one peroxide compound present in a synergistically microbicidally effective combined amount to control the growth of at least one contaminant microorganism in the fermentable carbohydrate-containing feedstock. Though not desiring to be bound to any theory, the peroxide compound may act as a scavenger to allow monochloramine to have a greater impact per unit time without adversely impacting yeast health in fermentation processes. As other advantages, the combined use of monochloramine and peroxide compound in an ethanol fermentation process can provide elimination or reduction in antibiotics in fermentation, reduction in lactic acid and acetic acid production in fermentation, increased yeast cell growth, viability, budding and vitality in fermentation, increased ethanol production in corn ethanol production, improved plant runnability, reduced production cost, increased value of dried distiller's grains for animal feed in corn ethanol production, and/or other improvements, or any combinations of these improvements.

The present invention provides an aqueous solution or formulation, which can be used in methods of this invention, which has a) monochloramine and b) at least one peroxide present in a combined amount synergistically effective to control the growth of at least one microorganism. The term “aqueous solution” as used herein can, as an example, refer to a solution that is predominantly water (e.g., over 50% by volume water such as over 75% by volume, over 95% by volume, or over 99% by volume water) and retains the solution characteristics of water. Where the aqueous solution contains solvents in addition to water, water is typically the predominant solvent.

The pH of the aqueous solution can be from about 4 to about 12, such as from about from about 4 to about 11, or from about 4 to about 10, or from about 4 to about 9, or from about 4 to about 8, or from about 4 to about 7, or from about 4 to about 6, or from about 5 to about 11, or from about 7 to about 10, or from 7.1 to 12, or from 7.5 to 10, or from 8 to 10. The aqueous solution can further include least one pH control agent, such as at least one acid or at least one base, or the aqueous solution may not include a pH control agent. If a pH control agent is included, the aqueous solution can include at least one acid, such as sulfuric acid and/or other acid, or at least one base, such as sodium hydroxide and/or other base. The addition or presence of at least one base along with the monochloramine and peroxide compound in the aqueous solution can provide optimal control of pathogenic bacteria. Some industrial processes involve lower pH conditions in an aqueous system during at least part of the process, such as ethanol fermentation processes, which can perform fermentation at a lower pH (e.g., about 4 to about 5.5). Fermentable carbohydrate-containing feedstock which can be used for ethanol fermentation can have a pH of from about 4 to about 12, or from about 4 to about 7. Before reaching the fermentation vessel, the fermentable carbohydrate-containing feedstock can be treated with an aqueous solution that combines the monochloramine and peroxide compound in a synergistically effective combined amount (and optionally with at least one base or pH control agent) to control the growth of at least one unwanted microorganism in the feedstock. This pre-fermentation treatment can be performed, with or without pH adjustment, on the fermentable carbohydrate-containing feedstock in piping, in process units or equipment, or in combinations of these, upstream (in advance) of the vessel(s) in which fermentation is performed (e.g., before where fermentation yeast and nutrients are introduced and combined with the fermentable carbohydrate-containing feedstock).

In lieu of adding the aqueous solution of the present invention to a material or medium to be treated, the monochloramine and peroxide compound, such as hydrogen peroxide, and, if used, at least one base, can be separately added to the product, material, or medium to be treated, such as indicated for ethanol fermentation processes. If separately added, these components are individually added so that the final amount of the mixture of monochloramine and peroxide compound at the time of use can preferably be that amount synergistically effective to control the growth of at least one microorganism in the treated product, material, or medium. In an ethanol fermentation process, the peroxide compound and monochloramine can be added separately to fermentable carbohydrate-containing feedstock or other process fluid in a holding vessel, or separately in piping, or separately in both of these process equipment or other process units or equipment, located in advance of the fermentation vessel. Alternatively or in addition, the peroxide compound and monochloramine can be added directly into the fermentation vessel, or after the fermentation vessel, or any combinations of these different introduction points.

The combined use of a) monochloramine and b) at least one peroxide compound in an aqueous solution is useful in preserving various type of products, media, or materials susceptible to attack by at least one microorganism. In the present invention, aqueous solutions comprising a) monochloramine and b) at least one peroxide compound (and optionally at least one base or pH control agent) are useful in preserving or controlling the growth of at least one microorganism in various types of industrial and/or food products, media, or materials susceptible to attack by microorganisms. The material or medium can be in the form of a solid, a dispersion, an emulsion, a mash, a slurry, or a solution. Such media or materials include, but are not limited to, for example, fermentation media/materials (as indicated), dyes, pastes, lumber, leathers, textiles, pulp, wood chips, tanning liquor, paper mill liquor, fiberglass, dairy processing, poultry processing, meat processing (e.g., beef, pork, lamb, or chicken), meat packing plant, animal slaughter houses, polymer emulsions, paints, paper and other coating and sizing agents, metalworking fluids, geological drilling lubricants, petrochemicals, cooling water systems, recreational water, influent plant water, waste water, pasteurizers, retort cookers, pharmaceutical formulations, cosmetic formulations, and toiletry formulations.

The combined use of a) monochloramine and b) at least one peroxide compound (and optionally the at least one base or pH control agent) in aqueous solutions can also be used to treat or preserve materials and media that include, but are not limited to, for example, fermentable carbohydrate-containing mashes or solutions (as indicated), wood pulp, wood chips, lumber, adhesives, coatings, animal hides, paper mill liquors, pharmaceutical formulations, cosmetic formulations, toiletry formulations, geological drilling lubricants, petrochemicals, agrochemical compositions, paints, leathers, plastics, seeds, plants, wood, metalworking fluids, cooling water, recreational water, influent plant water, waste water, pasteurizers, retort cookers, tanning liquors or solutions, starch, proteinaceous materials, acrylic latex paint emulsions, and textiles.

The combined use of a) monochloramine and b) at least one peroxide compound (and optionally at least one base or pH control agent) in aqueous solutions can be used to treat or preserve aqueous systems, such as ones subject to microbiological growth, attack, and degradation. These aqueous systems may be or include, but are not limited to, fresh, brackish or saltwater systems. Exemplary aqueous systems include, but are not limited to, latexes, surfactants, dispersants, stabilizers, thickeners, adhesives, starches, waxes, proteins, emulsifying agents, cellulose products, metal working fluids, cooling water, waste water, aqueous emulsions, aqueous detergents, coating compositions, paint compositions, and resins formulated in aqueous solutions, emulsions or suspensions. Additionally, with the present invention, microbiological deterioration of aqueous systems can be prevented or controlled including, but not limited to, related water-handling system, which may include cooling towers, pumps, heat exchangers, and pipelines, heating systems, scrubbing systems, and other similar systems, and the like.

The combined use of a) monochloramine and b) at least one peroxide compound (and optionally the at least one base or pH control agent) in aqueous solutions can also be used to protect or treat or preserve foods and/or surfaces in contact with food, such as fresh foods (e.g., vegetables and fruits) or meats, or dairy products or processing, for instance, to extend shelf life. The present invention can be used to protect or treat facilities that process food (meats, fruits, vegetables) including but not limited to the surfaces and machinery and devices that come into contact with the food or animal.

The combined use of a) monochloramine and b) at least one peroxide compound (and optionally at least one base or pH control agent) in aqueous solutions can also be useful in agrochemical formulations for the purpose of protecting seeds or crops against microbial spoilage.

According to the methods of the present invention, controlling or inhibiting the growth of at least one microorganism includes the reduction and/or the prevention of such growth.

It is to be further understood that by “controlling” (i.e., preventing) the growth of at least one of microorganism, the growth of the microorganism is inhibited. In other words, there is no growth or essentially no growth of the microorganism. “Controlling” the growth of at least one microorganism maintains the microorganism population at a desired level, reduces the population to a desired level (even to undetectable limits, e.g., zero population), and/or inhibits the growth of the microorganism. Thus, in the present invention, the products, material, or media susceptible to attack by the at least one microorganism can be preserved from this attack and the resulting spoilage and other detrimental effects caused by the microorganism. Further, it is also to be understood that “controlling” the growth of at least one microorganism also includes biostatically reducing and/or maintaining a low level of at least one microorganism such that the attack by the microorganism and any resulting spoilage or other detrimental effects are mitigated, i.e., the microorganism growth rate or microorganism attack rate is slowed down and/or eliminated.

When two chemical microbicides are mixed and added to the product, or added separately, three results are possible:
1) The chemicals in the product would produce an additive (neutral) effect.
2) The chemicals in the product would produce an antagonistic effect, or
3) The chemicals in the product would produce a synergistic effect.
   An additive effect has no economic advantage over the individual components. The antagonistic effect would produce a negative impact. Only a synergistic effect, which is less likely than either an additive or antagonistic effect, would produce a positive effect and therefore possess economic advantages.

It is known in the microbicidal literature that there is no theoretical method to anticipate additive, antagonistic, or synergistic effects when two biocides are mixed to yield a new formulation. Nor is there a method to predict the relative proportions of the different biocides required to produce one of the three effects described above.

Thus, the combination of a) monochloramine and b) at least one peroxide compound (and optionally at least one base or pH control agent) in aqueous solutions preferably achieve superior, i.e. greater than additive, microbicidal activity, even at low concentrations, against a wide variety of microorganisms. Examples of these microorganisms include fungi, bacteria, algae, and mixtures thereof, such as, but not limited to, for example, Lactobacillus, Pediococcus, Leuconostoc and Weissella species, Acetobacter sp., Trichoderma viride, Aspergillus niger, Pseudomonas aeruginosa, Enterobacter aerogenes, Klebsiella pneumoniae, and Chlorella sp. The microorganism can be an unwanted bacterium or bacteria. The unwanted bacteria can be unwanted bacteria in ethanol fermentation, such as Lactobacillus, Pediococcus, Leuconostoc and Weissella species, Acetobacter sp., or others. The combination of a) monochloramine and b) at least one peroxide compound of the present invention can have a low toxicity.

The monochloramine (NH₂Cl) (also referred to here as MCA) can be obtained or made on site. In dilute aqueous solution, chloramine is prepared by the reaction of ammonia with sodium hypochlorite:

NH₃+OCl⁻→NH₂Cl+HO^{-.}

This is also the first step of the Raschig hydrazine synthesis. The reaction is carried out in a slightly alkaline medium (pH 8.5 to 11). The acting chlorinating agent in this reaction is hypochloric acid (HOCl), which has to be generated by protonation of hypochlorite, and then reacts in a nucleophilic substitution of the hydroxo against the amino group. The reaction occurs quickest at around pH 8. At higher pH values the concentration of hypochloric acid is lower, at lower pH values ammonia is protonated to form ammonium ions NH₄⁺, which do not react further. The chloramine solution can be concentrated by vacuum distillation and by passing the vapor through potassium carbonate which absorbs the water. Chloramine can be extracted with ether. Gaseous chloramine can be obtained from the reaction of gaseous ammonia with chlorine gas (diluted with nitrogen gas):

2 NH₃(g)+Cl₂(g)⇔NH₂Cl(g)+NH₄Cl(s)

Pure chloramine can be prepared by passing fluoroamine through calcium chloride:

2NH₂F+CaCl_{2→}2NH₂Cl+CaF₂.

Methods for in situ chloramine generation are known which can be adapted for use in the method of the present invention. For example, rather than adding pure chloramine to the product, material, or system, sodium hypochlorite solution or chlorine can be added together with ammonia or ammonium salts to generate chloramine in situ prior to or at the time of combining with the peroxide compound. A single type of chloramine or combinations of different chloramines can be used.

“Peroxide compound” refers to compounds which can be hydroperoxide, organic peroxide, inorganic peroxide, peroxy-releasing compound, or any combinations thereof. A hydroperoxide can have the structure R—O—O—H, wherein R is a hydrogen or straight, branched and/or cyclic alkyl radical having 1 to 20 carbons atoms and can be optionally interrupted by one or more oxygen and/or carbonyl groups. An organic peroxide can have the structure R′—O—O—R″, wherein R′ and R″ are independently straight, branched, and/or cyclic alkyl radical having 1 to 20 carbons atoms and can be optionally interrupted by one or more oxygen and/or carbonyl groups. An inorganic peroxide can be selected from alkali metal peroxide, alkaline earth metal peroxide, transition metal peroxide, or any combinations thereof. A peroxygen-releasing compound can be selected from alkali metal percarbonates, alkaline earth metal percarbonates, transition metal percarbonates, alkali metal perborates, alkaline earth metal perborates, transition metal perborates, or any combinations thereof. The peroxide compound can be or include hydrogen peroxide (H₂O₂). Mixtures of peroxide compounds may be used, e.g., hydrogen peroxide and a different peroxide compound.

A peroxide compound can be more stable or can function better in an acidic environment, whereas MCA can function better in alkaline environment. As indicated, since some industrial processes, such as ethanol fermentation steps are usually performed under acidic pH conditions, any adjustment of aqueous solution pH to 7 or higher as part of treatment of a medium, if done as an option, preferably is performed at least in part or entirely before the fermentation step. Fermentation yeasts may not tolerate pH much below pH 3-4 or above 8-8.2 without adversely impacting the fermentation process. Ethanol fermentation processes can be treated with the aqueous solution of the present invention which combines peroxide compound and monochloramine with any addition of pH control agent managed to reduce or avoid adverse impacts on yeast or other components of the fermentation process.

At least one base can be present or included in the aqueous solution, as an option, to adjust, e.g., fine-tune, the pH for optimal effect or synergy between the peroxide compound and MCA from a biocidal efficiency standpoint, or cost perspective, or both. Any base can be used herein as a pH-adjusting adjunct for adjusting the pH (e.g., increasing pH). The base can be an alkali metal hydroxide, alkaline earth metal hydroxide, or any combination thereof. The base can be sodium hydroxide, potassium hydroxide, calcium hydroxide, barium hydroxide, magnesium hydroxide, sodium carbonate, or any combination thereof. Preferred bases for pH adjustment can include water-soluble alkalis such as sodium hydroxide, potassium hydroxide, or mixtures thereof. The base can be used as an aqueous solution. The base can be added to the aqueous solution before treatment of a product, material or medium, and/or can be added to the product, material or medium before or after treatment with the aqueous solution, or both. As indicated, the base can be used as a pH control agent. If desirable to reduce the pH, such as, e.g., to provide or maintain a pH of no greater than 12 or other pH value with a pH range of about 4 to about 12 (e.g., about 4 to 11, or 4 to 10, or 4 to 9, or 4 to 8, or 4 to 7, or 4 to 6, or other values), a pH control agent can used which is at least one acid. The at least one acid, if used, can be acetic acid, citric acid, hydrochloric acid, sulfuric acid, or other acids, or alum, or any combination thereof.

The amount of base, if added, can be an amount which adjusts the aqueous solution to a desired pH value or range. The concentration of the base can be any commercially available concentration (e.g., 0.1 N or 0.01N, or concentrated base) and/or can be diluted to any desired or appropriate concentration. The base can be present in a concentration so that the aqueous solution has a pH of from about 4 to about 12, or from about 5 to about 12, or from about 6 to about 11, or from about 7 to about 10, or from about 7.1 to about 9.9, or from about 7.5 to about 9.5, or from about 8 to about 9, or other values.

The aqueous solution to which the at least one base can be added can be a reservoir or flowing stream of aqueous fluid which already contains monochloramine but not yet peroxide compound. For instance, after the at least one base is added to an aqueous fluid comprising monochloramine, the resulting base-treated aqueous fluid can be further modified by addition of the peroxide compound before the aqueous fluid comprising all three components is introduced into an aqueous system (or product, material, or medium) to be treated. As another option, aqueous fluid comprising the monochloramine and the least one base can be added to the aqueous system (or product, material, or medium) to be treated, and the peroxide compound can be separately added to the aqueous system upstream or downstream thereof. As another option, the at least one base, monochloramine, and peroxide compound can be separately added to an aqueous system (or product, material, or medium) to be treated, wherein the aqueous solution is essentially prepared concurrent with treatment by it. The aqueous system or medium that can be treated in any of these manners with the aqueous solution can be aqueous fluid (e.g., water alone, or water-predominant solutions, or other water-based solutions) held in a pool, vessel, or flowing aqueous fluid in a conduit or open flowing stream, or other aqueous systems. The liquid system or medium may be an animal water trough or gutter through which drinking water flows or stands. As stated, the present invention also embodies the separate addition of the monochloramine and at least one peroxide compound, such as peroxide compound, and, if used, the at least one base or pH control agent, to products, materials, or media. According to this option, the components are individually added to the products, materials, or media so that the final amount of each component present at the time of use that can preferably be that amount synergistically effective, to control the growth of at least one microorganism.

The monochloramine and at least one peroxide compound, and, if used, the at least one base or pH control agent, can be added separately to the product, material, or medium, or system or environment that contains the product, material or medium. When adding separately, each of the monochloramine and peroxide compound, and, if used, the at least one base or pH control agent, can be added simultaneously, almost simultaneously (within 0.1 sec to 5 minutes of each other, for instance within 5 seconds, within 10 seconds, within 30 seconds, within 1 minute, within 2 minutes, within 5 minutes, or within 10 minutes of each other), or in sequence and in any order (e.g., peroxide compound first or monochloramine first). Further, in this option or in any embodiment of the present invention, the monochloramine can be formed in-situ in the presence of (or just before the MCA contacts) the product, material, or medium being treated or protected. The in-situ formation of the monochloramine can be done before or after peroxide compound is present. After adding (or forming) each of the monochloramine and peroxide compound, and, if used, the at least one base or pH control agent, in a liquid solution, medium or environment, mixture or agitation can be optionally used to mix the two (or three) components together for any amount of time (e.g., 1 second to 10 minutes or more). Each component can be applied by spraying, misting, coating, dipping, or any other technique/application that permits the contacting of the product, material, medium or system with each of a) monochloramine and b) at least one peroxide compound.

The microbicides in the aqueous solution of this invention may be used “as is” or may first be formulated with a solvent or a solid carrier. Suitable solvents include, for example, water; glycols, such as ethylene glycol, propylene glycol, diethylene glycol, dipropylene glycol, polyethylene glycol, and polypropylene glycol; glycol ethers; alcohols, such as methanol, ethanol, propanol, phenethyl alcohol and phenoxypropanol; ketones, such as acetone and methyl ethyl ketone; esters, such as ethyl acetate, butyl acetate, triacetyl citrate, and glycerol triacetate; carbonates, such as propylene carbonate and dimethyl carbonate; and mixtures thereof. The solvent can be selected from water, glycols, glycol ethers, esters and mixtures thereof. Hydrogen peroxide can be used in commercially available or synthesized forms as a solution in water, such as in concentrations of from about 3 wt. % to about 98 wt. %, or from about 10 wt. % to about 75 wt. %, or from about 20 wt. % to about 60 wt. %, or from about 30 wt. % to about 50 wt. %, or about 35 wt. % to about 45 wt. %, or about 40 wt. %, or other concentrations. Suitable solid carriers include, for example, cyclodextrin, silicas, diatomaceous earth, waxes, cellulosic materials, alkali and alkaline earth (e.g., sodium, magnesium, potassium) metal salts (e.g., chloride, nitrate, bromide, sulfate) and charcoal.

The components (a) monochloramine (MCA) and (b) at least one peroxide compound (and optionally the at least one base or pH control agent) also can be formulated in the form of a dispersion. The solvent component of the dispersion can be an organic solvent or water. Such dispersions can contain adjuvants, for example, co-solvents, thickeners, anti-freeze agents, dispersants, fillers, pigments, surfactants, biodispersants, sulfosuccinates, terpenes, furanones, polycations, stabilizers, scale inhibitors and/or anti-corrosion additives.

When components (a) monochloramine (MCA) and (b) at least one peroxide compound (and optionally the at least one base or pH control agent) are formulated in a solvent, the formulation may optionally contain surfactants. When such formulations contain surfactants, they are generally in the form of emulsive concentrates, emulsions, microemulsive concentrates, or microemulsions. Emulsive concentrates form emulsions upon the addition of a sufficient amount of water. Microemulsive concentrates form microemulsions upon the addition of a sufficient amount of water. Such emulsive and microemulsive concentrates are generally well known in the art; it is preferred that such formulations are free of surfactants. U.S. Pat. No. 5,444,078 may be consulted for further general and specific details on the preparation of various microemulsions and microemulsive concentrates.

For purposes of the present invention, the formulation of the present invention can be in the absence of other microbicides, and/or in the absence of metal containing compounds, and/or in the absence of organic acids, and/or in the absence of antibiotics, and/or in the absence of surfactants, and/or in the absence of any actives other than monochloramine and peroxide compound.

As described above, components (a) monochloramine (MCA) and (b) at least one peroxide compound (and optionally the at least one base or pH control agent) are preferably used in aqueous solution in synergistically effective amounts. The weight ratios of (a) to (b) vary depending on the type of microorganisms and product, material, or media to which the aqueous solution is applied. In view of the present invention, one skilled in the art can readily determine, without undue experimentation, the appropriate weight ratios for a specific application. The weight ratio (wt:wt basis) of component (a) to component (b) used in aqueous solution or formulation ranges from 1:1000 to 1000:1 (0.001:1 to 1:0.001), or from 1:99 to 99:1, or from 1:50 to 50:1, or from 1:40 to 40:1, or from 1:30 to 30:1, or from 1:20 to 20:1, or from 1:10 to 10:1, or from 1:5 to 5:1, or from 1:4 to 4:1, or from 1:3 to 3:1, or from 1:2.5 to 2.5:1, or from 1:2 to 2:1, or about from 1:1.5 to 1.5:1, or from about 1:1.25 to 1.25:1, or from about 1:1.1 to 1.1:1, or from about 1:10 to 1:1, or from about 1:7.5 to 1:1, or from about 1:5 to 1:1, or from 1:5 to 1:2, or from about 1:4 to 1:2, or from about 1:5 to 1:3. These weight ratios can be for the aqueous solution to be treated and/or can be the weight ratios of the aqueous solution prepared and used to treat an aqueous solution.

For instance, in an aqueous solution or formulation, the MCA can be present at a concentration of from 0.1 ppm to 50,000 ppm, or from 0.1 ppm to 10,000 ppm, or from 0.1 ppm to 5,000 ppm, or from 0.1 ppm to 1,000 ppm, or from 0.1 ppm to 750 ppm, or from 0.1 ppm to 500 ppm, or from 0.1 ppm to 250 ppm, or from 0.1 ppm to 100 ppm, or from 0.1 ppm to 75 ppm, or from 0.1 ppm to 50 ppm, or from 1 ppm to 5,000 ppm, or from 1 ppm to 1,000 ppm, or from 1 ppm to 750 ppm, or from 1 ppm to 450 ppm, or from 1 ppm to 250 ppm, or from 5 ppm to 250 ppm, or from 10 ppm to 250 ppm, or from 15 ppm to 250 ppm, or from 20 ppm to 250 ppm, or from 25 ppm to 250 ppm, or from 1 ppm to 225 ppm, or from 1 ppm to 200 ppm, or from 1 ppm to 175 ppm, or from 1 ppm to 150 ppm, or from 1 ppm to 100 ppm, or from 1 ppm to 75 ppm, or from 1 ppm to 50 ppm, or from 5 ppm to 150 ppm, or from 10 ppm to 150 ppm, or from 15 ppm to 150 ppm, or from 20 ppm to 150 ppm, or from 25 ppm to 150 ppm, or from 50 ppm to 150 ppm, or from 5 ppm to 125 ppm, or from 5 ppm to 100 ppm, or from 5 ppm to 75 ppm, or from 5 ppm to 50 ppm, or from 10 ppm to 100 ppm, or from 15 ppm to 100 ppm, or from 20 ppm to 100 ppm, or from 25 ppm to 100 ppm, or from 50 ppm to 100 ppm, or from 10 ppm to 90 ppm, or from 10 ppm to 75 ppm, or from 10 ppm to 50 ppm, or from 10 ppm to 25 ppm, or from 15 ppm to 80 ppm, or from 25 ppm to 80 ppm, or from 15 ppm to 75 ppm, or from 15 ppm to 60 ppm, or from 15 ppm to 50 ppm, or from 20 ppm to 60 ppm, or from 25 ppm to 50 ppm, and the peroxide compound can be present at a concentration of 0.1 ppm to 50,000 ppm, from 0.1 ppm to 10,000 ppm, or from 0.1 ppm to 5,000 ppm, or from 0.1 ppm to 1,000 ppm, or from 0.1 ppm to 750 ppm, or from 0.1 ppm to 500 ppm, or from 0.1 ppm to 250 ppm, or from 0.1 ppm to 100 ppm, or from 0.1 ppm to 75 ppm, or from 0.1 ppm to 50 ppm, or from 1 ppm to 5,000 ppm, or from 1 ppm to 1,000 ppm, or from 1 ppm to 750 ppm, or from 1 ppm to 450 ppm, or from 5 ppm to 450 ppm, or from 1 ppm to 350 ppm, or from 5 ppm to 350 ppm, or from 1 ppm to 250 ppm, or from 5 ppm to 250 ppm, or from 10 ppm to 250 ppm, or from 15 ppm to 250 ppm, or from 20 ppm to 250 ppm, or from 25 ppm to 250 ppm, or from 1 ppm to 225 ppm, or from 1 ppm to 200 ppm, or from 1 ppm to 175 ppm, or from 1 ppm to 150 ppm, or from 1 ppm to 100 ppm, or from 1 ppm to 75 ppm, or from 1 ppm to 50 ppm, or from 5 ppm to 150 ppm, or from 10 ppm to 150 ppm, or from 15 ppm to 150 ppm, or from 20 ppm to 150 ppm, or from 25 ppm to 150 ppm, or from 50 ppm to 150 ppm, or from 5 ppm to 125 ppm, or from 5 ppm to 100 ppm, or from 5 ppm to 75 ppm, or from 5 ppm to 50 ppm, or from 10 ppm to 125 ppm, or from 15 ppm to 125 ppm, or from 20 ppm to 125 ppm, or from 25 ppm to 125 ppm, or from 50 ppm to 125 ppm, or from 10 ppm to 100 ppm, or from 10 ppm to 75 ppm, or from 10 ppm to 50 ppm, or from 15 ppm to 90 ppm, or from 20 ppm to 90 ppm, or from 25 ppm to 90 ppm, or from 25 ppm to 70 ppm, or from 25 ppm to 60 ppm, or from 25 ppm to 90 ppm, or from 25 ppm to 75 ppm, or from 25 ppm to 50 ppm. These ppm concentrations can be for an aqueous solution to be treated and/or can be the ppm concentrations of the aqueous solution prepared and used to treat an aqueous solution.

These dosages and others described herein can be calculated or measured values or can be considered residual ppm amounts present in the aqueous solution being treated.

As one precise option in the present invention, in an aqueous solution or formulation, the MCA can be present at a concentration of from 30 ppm to 100 ppm or from 50 ppm to 100 ppm, and the peroxide compound can be present at a concentration of 75 ppm to 125 ppm or below 200 ppm. These amounts especially permit an increase in bacterial count control in manner that would not be achievable if the MCA or peroxide were used by themselves at the same concentrations.

In general, for the aqueous solution having a pH of from about 4 to about 12 and, if used, base or pH control agent, a synergistically microbicidally effective response (e.g., fungicidal, bactericidal, or algicidal response) can be obtained when the combination of component (a) and component (b) is employed in concentrations ranging about 0.1 ppm to 5% (i.e., 50,000 ppm) of the MCA, preferably from 0.1 ppm to 750 ppm, more preferably from 1 ppm to 450 ppm, even more preferably from 1 ppm to 250 ppm, and most preferably from 1 ppm to 100 ppm; and from 0.1 ppm to 50,000 ppm of the peroxide compound (e.g., peroxide compound), preferably from 0.1 ppm to 750 ppm, more preferably 1 ppm to 450 ppm, even more preferably 1 ppm to 250 ppm, and most preferably 5 ppm to 100 ppm. In general, an effective fungicidal, bactericidal, or algicidal response can be obtained when the synergistic combination is employed in concentrations ranging about 0.1 ppm to 1% (i.e., 10,000 ppm) of the MCA, preferably 0.1 ppm to 750 ppm, more preferably 1 ppm to 450 ppm, and most preferably from 1 ppm to 100 ppm; and from about 0.1 ppm to 5,000 ppm of the peroxide compound (e.g., hydrogen peroxide), preferably 0.1 ppm to 750 ppm, more preferably 5 to 450 ppm, and most preferably, 5 ppm to 150 ppm. These ppm concentrations can be for the aqueous solution to be treated and/or can be the ppm concentrations of the aqueous solution prepared and used to treat an aqueous solution.

Depending upon the specific application, the aqueous solution can be prepared in liquid form by dissolving, dispersing, or in-situ forming the monochloramine and at least one peroxide compound, and, if used, at least one base or pH control agent, in water or other aqueous fluid. The preservative containing the aqueous solution of the present invention may be prepared in an emulsion form by emulsifying it in water, or if necessary, by adding a surfactant. Additional chemicals, such as insecticides, may be added to the foregoing preparations and aqueous solutions depending upon the intended use of the preparation.

The mode as well as the rates of application of the aqueous solution of this invention could vary depending upon the intended use. The aqueous solution could be applied by spraying or brushing onto the material or product. The material or product in question could also be treated by dipping in a suitable formulation of the aqueous solution. In a liquid or liquid-like medium, the aqueous solution could be added into the medium by pouring, or by metering with a suitable device so that a solution or a dispersion of the aqueous solution can be produced.

Fermentation systems which can be can be treated with the synergistic microbicidal combination of the present invention include systems for production of ethanol fermentation systems and pharmaceutical fermentations systems, or other fermentation systems. The ethanol fermentations systems can include those for corn ethanol, cane-to-ethanol, dry grind ethanol, wet grain ethanol, wheat-to-ethanol, barley-to-ethanol, oats-to-ethanol, rye-to-ethanol, sorghum-to-ethanol, cellulosic-to-ethanol, sugar beet-to-ethanol, rice-to-ethanol, or other ethanol fermentation systems.

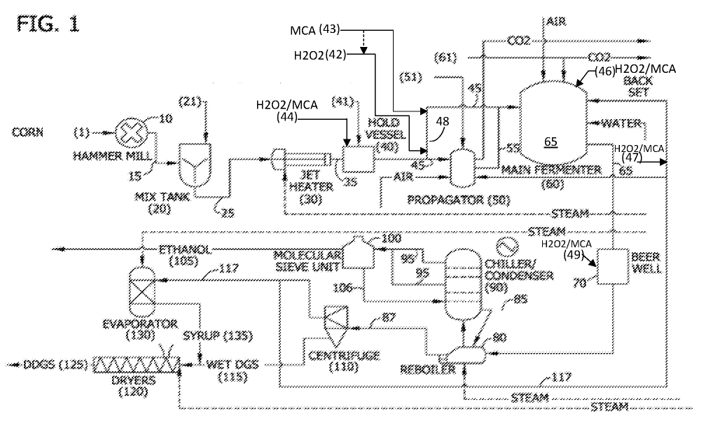

A method according to the present invention can be practiced in conventional ethanol production plants with modifications that can be easily made in view of the present invention. Referring to FIG. 1, a process for treating an ethanol fermentation system with the synergistic microbicidal combination of the present invention is generally shown as directed to introducing combinations of hydrogen peroxide or other peroxide compound and monochloramine (MCA) in combinations or to provide combinations thereof in the system via one or more of introduction locations (42), (43), (44), (46), (47), (49), wherein other exemplary features of the system, as an option, include (1) coarse milling (10) to generate milled corn (15), and the milled corn (15) can be combined with a-amylase or other liquefaction enzyme and water (21) in a mix tank (20) in a pre-liquefaction step to form pre-liquified corn mash (25). The pre-liquified corn (25) can be fed to a jet heater (30) for heat liquefaction to generate heat-liquified corn mash (35), and the heat-liquified corn mash (35) can be combined with a-amylase (and/or other liquefying enzyme) and water (41) in a holding vessel (40) in a digest step to generate liquified corn mash (45). A portion of the liquified corn mash (45) can be combined with glucoamylase and/or other saccharifaction enzyme, a nutrient source, yeast, and water (51) in a propagation tank (50) to generate pitching yeast (55), which can be fed to fermenter vessel (60). The rest of the liquefied corn mash (45) can be fed through piping (48) to fermenter vessel (60). This portion of the liquified corn (45) fed through piping (48), with the pitching yeast (55) and glucoamylase, a nitrogen-containing nutrient source and water (61) can be combined in the fermenter vessel (60) to generate a fermentation composition (65). The fermentation composition can be sent to a beer well (70), then to a reboiler (80) for recovery of crude ethanol (95) from the overhead stream (85) in a condenser (90). The crude ethanol (95) can be sent to a molecular sieve unit (100) for separation of ethanol (105) from byproducts (106). The stillage or reboiler bottoms (87) can be sent to a centrifuge (110) where wet distiller grain solids (“DGS”) (115) and centrifugate (117) (liquid-containing fraction) are separated. All or a portion of the centrifugate (117) can optionally be recycled to the mix tank (20), propagation tank (50) and/or fermenter (60) as backset. The centrifugate (117) that is not recycled can be fed to an evaporator (130) where it can be concentrated to produce syrup (135), and the syrup (135) can be combined with the wet DGS (115) and the combination is sent to a dryer (120) in which DDGS (125) can be prepared. In an alternative, wet DGS (115) can be dried in the absence of syrup (135) to generate dried distillers grain (“DDG”) (not shown). To simplify the illustration in FIG. 1, additional pumps, heat exchangers, and other conventional equipment that can be used in the process are not shown.

As shown in FIG. 1, as an option, the peroxide compound and monochloramine can be added in synergistically effective combined amounts at one or more locations (44), (42), (43), (47) before the fermentation vessel (60), (46) in the fermentation vessel (60), at one or more locations (49) after the fermentation vessel (60), or any combinations thereof. As a preferred option, the peroxide compound and monochloramine are added in a synergistically effective combined amount at least at one or more locations before the fermentation vessel (60) (e.g., at least at one or more of locations (44), (42), (43), (47)). The treatment can be performed, with or without pH adjustment, on the fermentable carbohydrate-containing feedstock before introduction to fermentation vessel (60) in holding vessel (40), piping (48), propagator (50) (not shown) or other process units/equipment (e.g., pumps), or in any combinations of these options. The peroxide compound and monochloramine can be added from the same aqueous solution into holding vessel (40), piping (48) (e.g., using 42 and alternate line shown in dashes for 43), or into the backset line (47), or any combinations of these or other process equipment located in advance of the fermentation vessel (60). The peroxide compound and monochloramine can be added separately to the fermentable carbohydrate-containing feedstock in holding vessel (40), or separately into piping (48) as shown by (42) and (43) in FIG. 1, or separately into the backset line (47), or any combinations of these or other process equipment located in advance of the fermentation vessel (60). The peroxide compound and monochloramine can be added from the same aqueous solution or separately (44) into holding vessel (40), and then additional monochloramine (43) can be added in piping (48) to the mash discharged from holding vessel (40). Mixing of separately introduced peroxide compound and monochloramine in the feedstock or other process fluid upstream of the fermentation vessel can be provided with turbulence present in process units by agitators therein, or by pumps, or in-line static mixers in piping, or by introducing the peroxide compound and monochloramine ahead of a bend or bends in the piping that encourage turbulence in the fluids passing through the piping, or other equipment arrangements or combinations of these. The combined peroxide compound and monochloramine may survive about 5 to about 10 minutes, or other periods of time, in the treated process system in the presence of an organic load (e.g., fermentable carbohydrate-containing feedstock). By adding the peroxide and monochloramine components to process fluid at a location or locations sufficiently near the fermentation vessel, from a temporal standpoint, before introduction into the fermentation vessel, control of lactic acid, acetic acid or both in the fermentation vessel during fermentation may be provided even if the peroxide compound and monochloramine are not added directly into the fermentation vessel (which is another option of the present invention). The peroxide compound and monochloramine can be introduced into the fermentation vessel (60) as indicated by (46) from a single aqueous solution or separately. The peroxide compound and monochloramine can be introduced into the fermented composition (65) after discharge from the fermentation vessel (60) as indicated by (49) (e.g., at a beer well as shown or other post-fermentation process unit/equipment or piping) from a single aqueous solution or separately.

Other aspects, equipment and details of the ethanol fermentation chemistry, process and system can be based on those used in ethanol production plants, such as those described in U.S. Pat. No. 8,951,960 and U.S. Patent Application Publication No. 2017/0107543, which are incorporated herein in their entireties by reference.

The microbicidal and synergistic activity of the combinations described above has been confirmed using standard laboratory techniques as illustrated below. The following examples are intended to illustrate, not limit, the present invention.

### EXAMPLE 1

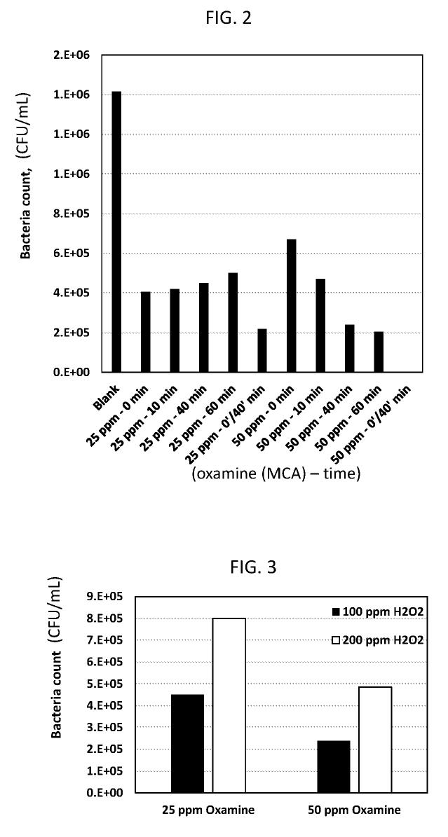

The effect of combined amounts of hydrogen peroxide and monochloramine on bacterial growth control and their addition timing was studied using laboratory experiments. To establish the effect of the combined addition and time between the addition of hydrogen peroxide and (“Oxamine”) on bacterial growth control, laboratory experiments were performed using hydrogen peroxide at a dosage of 100 ppm, monochloramine (“Oxamine”) at dosages of 25 ppm and 50 ppm, and a time between peroxide and monochloramine addition of 0, 10, 40 and 60 minutes, and a sequential monochloramine addition at 0 and 40 minutes (0′/40′) for 25 and 50 ppm dosages. All experiments were carried out with 35 wt % corn slurry solution. The results are shown in FIGS. 2 and 3.

The results in FIG. 2 show that hydrogen peroxide and 25 ppm monochloramine treatments led to same level of bacterial control (FIG. 2). For 50 ppm monochloramine treatments, there was a slight improvement in bacteria control for the time duration of 40 and 60 minutes. The time between peroxide and monochloramine addition did not show a significant effect on bacterial growth control for the laboratory scale of testing performed. The sequential monochloramine addition led to significant bacterial growth control which can provide synergistic microbicidal growth control. As indicated by the results in FIG. 3, increasing peroxide dosage from 100 to 200 ppm did not show improvement in bacterial growth control for the laboratory scale of testing performed.

### EXAMPLE 2

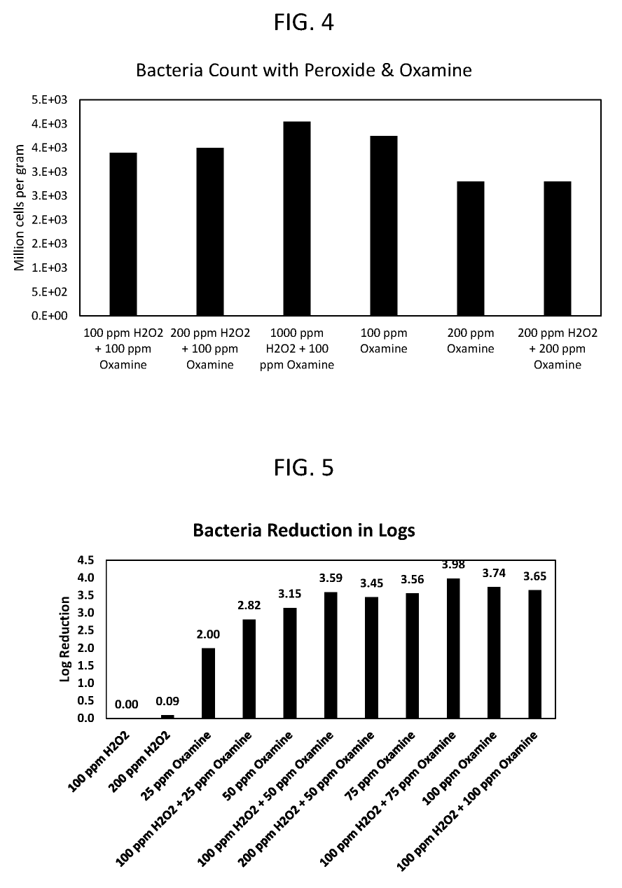

The effect of combined amounts of hydrogen peroxide and monochloramine at other tested addition amounts on bacterial growth control and their addition timing was studied using laboratory experiments. For this additional study, laboratory experiments were performed using hydrogen peroxide at dosages of 100 ppm, 200 ppm, and 1000 ppm, and monochloramine (“Oxamine”) at dosages of 100 ppm and 200 ppm. The time between peroxide and monochloramine addition was up to 10 minutes (i.e., 0-10 minutes). All experiments were carried out with 35 wt % corn slurry solution. The results are shown in FIG. 4.

The results in FIG. 4 show that for the samples treated with 100 ppm monochloramine and hydrogen peroxide, bacterial growth control was improved for the sample treated with hydrogen peroxide at concentration of 100 ppm with the 100 ppm monochloramine compared to the use of 100 ppm monochloramine alone. As indicated by the results in FIG. 4, increasing peroxide dosage from 100 to 200 ppm and 1000 ppm for the treatments done at 100 ppm monochloramine did not show improvement in bacterial growth control for the laboratory scale of testing performed. Treatment with 200 ppm peroxide and 200 ppm monochloramine showed comparable results with monochloramine alone at this dosage for 100× dilution (shown in FIG. 4).

The effect of combined amounts of hydrogen peroxide and monochloramine at other tested addition amounts on bacterial growth control and their addition timing was further studied using laboratory experiments. For this additional study, laboratory experiments were performed using hydrogen peroxide at dosages of 100 ppm and 200 ppm, and monochloramine (“Oxamine”) at dosages of 25 ppm, 50 ppm, 75 ppm, and 100 ppm. The time between peroxide and monochloramine addition was up to 10 minutes (i.e., 0-10 minutes). All experiments were carried out with 35 wt % corn slurry solution. The results are shown in Table 1 and FIG. 5.

TABLE 1

| | Bacteria | % |
|---|---|---|
| Treatment | Count | Reduction |
| Blank | 5.E+06 | |
| 100 ppm H2O2 | 5.E+06 | 0.00 |
| 200 ppm H2O2 | 4.E+06 | 18.37 |
| 25 ppm Oxamine | 5.E+04 | 98.99 |
| 100 ppm H2O2 + 25 ppm Oxamine | 8.E+03 | 99.85 |
| 50 ppm Oxamine | 4.E+03 | 99.93 |
| 100 ppm H2O2 + 50 ppm Oxamine | 1.E+03 | 99.97 |
| 200 ppm H2O2 + 50 ppm Oxamine | 2.E+03 | 99.96 |
| 75 ppm Oxamine | 1.E+03 | 99.97 |
| 100 ppm H2O2 + 75 ppm Oxamine | 5.E+02 | 99.99 |
| 100 ppm Oxamine | 9.E+02 | 99.98 |
| 100 ppm H2O2 + 100 ppm Oxamine | 1.E+03 | 99.98 |

The results in Table 1 and FIG. 5 show that the samples treated with 75 ppm monochloramine and 100 ppm hydrogen peroxide provided the greatest improvement in bacterial growth control. Bacterial growth control was improved for the sample treated with hydrogen peroxide at concentration of 100 ppm with the 100 ppm monochloramine compared to the use of 100 ppm monochloramine alone. As indicated by the results in Table 1 and FIG. 5, increasing peroxide dosage from 100 ppm to 200 ppm did not show improvement in bacterial growth control for the laboratory scale of testing performed. The present invention includes the following aspects/embodiments/features in any order and/or in any combination:
1. A method of controlling the growth of at least one microorganism in or on a product, material, or medium susceptible to attack by a microorganism, the method comprising treating the product, material or medium with aqueous solution comprising (a) monochloramine and (b) at least one peroxide compound, wherein components (a) and (b) are present in a synergistically microbicidally effective combined amount to control the growth of at least one microorganism.
2. The method of any preceding or following embodiment/feature/aspect, wherein the material or medium is fermentable mash or solution, wood pulp or paper, wood chips, lumber, paints, leathers, adhesives, coatings, animal hides, tanning liquor, paper mill liquor, fiberglass, dairy processing, poultry processing, meat packing facilities, meat processing, metalworking fluids, petrochemicals, pharmaceutical formulations, cooling water, recreational water, dyes, clays, mineral slurries, cationic surfactants, formulations with cationic surfactants, influent water, waste water, pasteurizers, retort cookers, cosmetic formulations, toiletry formulations, textiles, geological, drilling lubricants, or agrochemical compositions for crop or seed protection.
3. The method of any preceding or following embodiment/feature/aspect, wherein the microorganism is bacteria, fungi, algae or combinations thereof.
4. The method of any preceding or following embodiment/feature/aspect, wherein the material or medium is in the form of a solid, a dispersion, an emulsion, a mash, a slurry, or a solution.
5. A method to control growth of at least one contaminant microorganism in a fermentable carbohydrate-containing feedstock comprising contacting the fermentable carbohydrate-containing feedstock with (a) monochloramine and (b) at least one peroxide compound, wherein components (a) and (b) are present in a synergistically microbicidally effective combined amount to control the growth of at least one contaminant microorganism in the fermentable carbohydrate-containing feedstock.
6. The method of any preceding or following embodiment/feature/aspect, wherein the monochloramine is present in the fermentable carbohydrate-containing feedstock at a concentration of 0.1 ppm to 750 ppm, and the at least one peroxide compound is present in the fermentable carbohydrate-containing feedstock at a concentration of 0.1 ppm to 750 ppm.
7. The method of any preceding or following embodiment/feature/aspect, wherein the monochloramine is present in the fermentable carbohydrate-containing feedstock at a concentration of 1 ppm to 450 ppm, and the at least one peroxide compound is present in the fermentable carbohydrate-containing feedstock at a concentration of 5 ppm to 450 ppm.
8. The method of any preceding or following embodiment/feature/aspect, wherein the monochloramine and the at least one peroxide compound are added to the fermentable carbohydrate-containing feedstock in a weight ratio of 0.001:1 to 1:0.001.
9. The method of any preceding or following embodiment/feature/aspect, wherein the peroxide compound is hydroperoxide, organic peroxide, inorganic peroxide, peroxy-releasing compound, or any combinations thereof.
10. The method of any preceding or following embodiment/feature/aspect, wherein the microorganism is a bacterium.
11. The method of any preceding or following embodiment/feature/aspect, wherein the fermentable carbohydrate-containing feedstock comprises fermentable carbohydrate derived from cereal grain, cellulose, fruit, non-cereal grain vegetable, or any combinations thereof.
12. A method for producing ethanol by fermentation with controlled growth of contaminant microorganisms comprising:

a) adding (a) monochloramine and (b) at least one peroxide compound to fermentable carbohydrate-containing feedstock to provide treated feedstock, wherein components (a) and (b) are present in a synergistically microbicidally effective combined amount to control the growth of at least one contaminant microorganism in the treated feedstock;

b) fermenting the treated feedstock in the presence of yeast in a vessel to produce fermented mash comprising ethanol and a solids content; and

c) distilling the fermented mash to separate at least a portion of the ethanol from stillage comprising said solids content.
13. The method of any preceding or following embodiment/feature/aspect, wherein monochloramine and the at least one peroxide compound are added to the fermentable carbohydrate-containing feedstock before, after, or both before and after the feedstock is introduced into the fermenter vessel and present with the yeast.
14. The method of any preceding or following embodiment/feature/aspect, wherein monochloramine and the at least one peroxide compound are added to the fermentable carbohydrate-containing feedstock before the treated feedstock is introduced into the fermenter vessel and combined with the yeast.
15. The method of any preceding or following embodiment/feature/aspect, wherein at least a portion of the at least one peroxide compound is added to the fermentable carbohydrate-containing feedstock before adding the monochloramine to the fermentable carbohydrate-containing feedstock.
16. The method of any preceding or following embodiment/feature/aspect, further comprising providing a holding vessel upstream of the fermenter vessel where the fermentable carbohydrate-containing feedstock is temporarily held before conducted through piping to the fermenter vessel, wherein the monochloramine and the at least one peroxide compound is added to the fermentable carbohydrate-containing feedstock in both the holding vessel and in the piping before introduced into the fermenter vessel.
17. The method of any preceding or following embodiment/feature/aspect, wherein the adding of the (a) monochloramine and the (b) at least one peroxide compound to the fermentable carbohydrate-containing feedstock is provided without reducing yeast population of yeast present in the vessel used for the fermenting.
18. The method of any preceding or following embodiment/feature/aspect, wherein the adding of the (a) monochloramine and the (b) at least one peroxide compound to the fermentable carbohydrate-containing feedstock reduces total lactic acid and acetic acid produced in the fermenting compared to fermenting in the absence of adding compounds (a) and (b) to the fermentable carbohydrate-containing feedstock.
19. The method of any preceding or following embodiment/feature/aspect, wherein the fermenting is performed in the absence of added antibiotic.
20. The method of any preceding or following embodiment/feature/aspect, wherein the fermentable carbohydrate-containing feedstock comprises flowable carbohydrate-containing feedstock derived from corn in an aqueous medium.
21. The method of any preceding or following embodiment/feature/aspect, wherein the microorganism is a bacterium. 22. The method of any preceding or following embodiment/feature/aspect, wherein the monochloramine is added to the fermentable carbohydrate-containing feedstock at a concentration of 0.1 ppm to 750 ppm, and the at least one peroxide compound is added to the fermentable carbohydrate-containing feedstock at a concentration of 0.1 ppm to 750 ppm.
23. The method of any preceding or following embodiment/feature/aspect, wherein the monochloramine is present in the fermentable carbohydrate-containing feedstock at a concentration of 1 ppm to 450 ppm, and the at least one peroxide compound is present in the fermentable carbohydrate-containing feedstock at a concentration of 5 ppm to 450 ppm.
24. The method of any preceding or following embodiment/feature/aspect, wherein the monochloramine and the at least one peroxide compound are added to the fermentable carbohydrate-containing feedstock in a ratio of 0.001:1 to 1:0.001.
25. The method of any preceding or following embodiment/feature/aspect, wherein the peroxide compound is hydroperoxide, organic peroxide, inorganic peroxide, peroxy-releasing compound, or any combinations thereof.
26. The method of any preceding or following embodiment/feature/aspect, wherein the peroxide compound is a hydroperoxide having the structure R—O—O—H, wherein R is a hydrogen or straight, branched and/or cyclic alkyl radical having 1 to 20 carbons atoms and can be optionally interrupted by one or more oxygen and/or carbonyl groups.
27. The method of any preceding or following embodiment/feature/aspect, wherein the peroxide compound is an organic peroxide having the structure R′—O—O—R″, wherein R′ and R″ are independently straight, branched, and/or cyclic alkyl radical having 1 to 20 carbons atoms and can be optionally interrupted by one or more oxygen and/or carbonyl groups.
28. The method of any preceding or following embodiment/feature/aspect, wherein the peroxide compound is an inorganic peroxide selected from alkali metal peroxide, alkaline earth metal peroxide, transition metal peroxide, or any combinations thereof.
29. The method of any preceding or following embodiment/feature/aspect, wherein the peroxide compound is a peroxygen-releasing compound selected from alkali metal percarbonates, alkaline earth metal percarbonates, transition metal percarbonates, alkali metal perborates, alkaline earth metal perborates, transition metal perborates, or any combinations thereof.
30. The method of any preceding or following embodiment/feature/aspect, wherein the pH of the fermentable carbohydrate-containing feedstock is from about 4 to about 7.
31. The method of any preceding or following embodiment/feature/aspect, further comprising the steps of:

d) separating the stillage into a liquids-containing fraction and a solids-containing fraction;

e) optionally recycling at least portion of the liquids-containing fraction of d) into the fermenter vessel;

f) recovering the solids-containing fraction of d) with drying of at least a portion of the solids-containing fraction to produce evaporated vapors and distillers dried grains product free of antibiotics.
32. An aqueous solution comprising (a) monochloramine and (b) at least one peroxide compound, wherein components (a) and (b) are present in a synergistically microbicidally effective combined amount to control the growth of at least one microorganism.
33. The aqueous solution of any preceding or following embodiment/feature/aspect, wherein the monochloramine is present in the aqueous solution at a concentration of 0.1 ppm to 750 ppm, and the at least one peroxide compound is present in the aqueous solution at a concentration of 0.1 ppm to 750 ppm.
34. The aqueous solution of any preceding or following embodiment/feature/aspect comprising the monochloramine is present in the aqueous solution at a concentration of 1 ppm to 450 ppm, and the at least one peroxide compound is present in the aqueous solution at a concentration of 5 ppm to 450 ppm.
35. The aqueous solution of any preceding or following embodiment/feature/aspect, wherein the monochloramine and the at least one peroxide compound are added to the aqueous solution in a ratio of 0.001:1 to 1:0.001.
36. The aqueous solution of any preceding or following embodiment/feature/aspect, wherein the peroxide compound is hydroperoxide, organic peroxide, inorganic peroxide, peroxy-releasing compound, or any combinations thereof.
37. The method or aqueous solution or formulation for any preceding or following embodiment/feature/aspect wherein said monochloramine and said peroxide compound are present in a synergistically microbicidally effective combined amount to control the growth of at least one microorganism, wherein said synergistically microbicidally effective combined amount is demonstrated by a formula of Q_{A}/Qₐ+Q_{B}/Q_{b}, wherein
Qₐ=Concentration of compound A in parts per million, acting alone, which produced an end point to completely prevent growth of a bacteria,
Q_{b}=Lowest concentration of compound B in parts per million, acting alone, which produced an end point to completely prevent growth of said bacteria,
Q_{A}=Lowest concentration of compound A in parts per million, in the mixture, which produced an end point to completely prevent growth of said bacteria,
Q_{B}=Lowest concentration of compound B in parts per million, in the mixture, which produced an end point to completely prevent growth of said bacteria,
   and where the sum of Q_{A}/Qₐ and Q_{B}/Q_{b} is less than one, and wherein said bacteria is Pseudomonas aeruginosa or Enterobacter aerogenes.

The present invention can include any combination of these various features or embodiments above and/or below as set forth in sentences and/or paragraphs. Any combination of disclosed features herein is considered part of the present invention and no limitation is intended with respect to combinable features.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the specification and practice of the invention disclosed herein. It is intended that the specification and examples be considered exemplary only, with a true scope and spirit of the invention being indicated by the following claims.

What is claimed is:

1. A method of controlling the growth of at least one microorganism in or on a product, material, or medium susceptible to attack by a microorganism, the method comprising treating the product, material or medium with aqueous solution comprising (a) monochloramine and (b) at least one peroxide compound, wherein components (a) and (b) are present in a synergistically microbicidally effective combined amount to control the growth of at least one microorganism.

2. The method of claim 1, wherein the material or medium is fermentable mash or solution, wood pulp or paper, wood chips, lumber, paints, leathers, adhesives, coatings, animal hides, tanning liquor, paper mill liquor, fiberglass, dairy processing, poultry processing, meat packing facilities, meat processing, metalworking fluids, petrochemicals, pharmaceutical formulations, cooling water, recreational water, dyes, clays, mineral slurries, cationic surfactants, formulations with cationic surfactants, influent water, waste water, pasteurizers, retort cookers, cosmetic formulations, toiletry formulations, textiles, geological, drilling lubricants, or agrochemical compositions for crop or seed protection.

3. The method of claim 1, wherein the microorganism is bacteria, fungi, algae or combinations thereof.

4. The method of claim 1, wherein the material or medium is in the form of a solid, a dispersion, an emulsion, a mash, a slurry, or a solution.

5. A method to control growth of at least one contaminant microorganism in a fermentable carbohydrate-containing feedstock comprising contacting the fermentable carbohydrate-containing feedstock with (a) monochloramine and (b) at least one peroxide compound, wherein components (a) and (b) are present in a synergistically microbicidally effective combined amount to control the growth of at least one contaminant microorganism in the fermentable carbohydrate-containing feedstock.

6. The method of claim 5, wherein the monochloramine is present in the fermentable carbohydrate-containing feedstock at a concentration of 0.1 ppm to 750 ppm, and the at least one peroxide compound is present in the fermentable carbohydrate-containing feedstock at a concentration of 0.1 ppm to 750 ppm.

7. The method of claim 5, wherein the monochloramine is present in the fermentable carbohydrate-containing feedstock at a concentration of 1 ppm to 450 ppm, and the at least one peroxide compound is present in the fermentable carbohydrate-containing feedstock at a concentration of 5 ppm to 450 ppm.

8. The method of claim 5, wherein the monochloramine and the at least one peroxide compound are added to the fermentable carbohydrate-containing feedstock in a weight ratio of 0.001:1 to 1:0.001.

9. The method of claim 5, wherein the peroxide compound is hydroperoxide, organic peroxide, inorganic peroxide, peroxy-releasing compound, or any combinations thereof.

10. The method of claim 5, wherein the microorganism is a bacterium.

11. The method of claim 5, wherein the fermentable carbohydrate-containing feedstock comprises fermentable carbohydrate derived from cereal grain, cellulose, fruit, non-cereal grain vegetable, or any combinations thereof.

12. A method for producing ethanol by fermentation with controlled growth of contaminant microorganisms comprising:
a) adding (a) monochloramine and (b) at least one peroxide compound to fermentable carbohydrate-containing feedstock to provide treated feedstock, wherein components (a) and (b) are present in a synergistically microbicidally effective combined amount to control the growth of at least one contaminant microorganism in the treated feedstock;
b) fermenting the treated feedstock in the presence of yeast in a vessel to produce fermented mash comprising ethanol and a solids content; and
c) distilling the fermented mash to separate at least a portion of the ethanol from stillage comprising said solids content.

13. The method of claim 12, wherein monochloramine and the at least one peroxide compound are added to the fermentable carbohydrate-containing feedstock before, after, or both before and after the feedstock is introduced into the fermenter vessel and present with the yeast.

14. The method of claim 13, wherein monochloramine and the at least one peroxide compound are added to the fermentable carbohydrate-containing feedstock before the treated feedstock is introduced into the fermenter vessel and combined with the yeast.

15. The method of claim 14, wherein at least a portion of the at least one peroxide compound is added to the fermentable carbohydrate-containing feedstock before adding the monochloramine to the fermentable carbohydrate-containing feedstock.

16. The method of claim 14, further comprising providing a holding vessel upstream of the fermenter vessel where the fermentable carbohydrate-containing feedstock is temporarily held before conducted through piping to the fermenter vessel, wherein the monochloramine and the at least one peroxide compound is added to the fermentable carbohydrate-containing feedstock in both the holding vessel and in the piping before introduced into the fermenter vessel.

17. The method of claim 12, wherein the adding of the (a) monochloramine and the (b) at least one peroxide compound to the fermentable carbohydrate-containing feedstock is provided without reducing yeast population of yeast present in the vessel used for the fermenting.

18. The method of claim 12, wherein the adding of the (a) monochloramine and the (b) at least one peroxide compound to the fermentable carbohydrate-containing feedstock reduces total lactic acid and acetic acid produced in the fermenting compared to fermenting in the absence of adding compounds (a) and (b) to the fermentable carbohydrate-containing feedstock.

19. The method of claim 12, wherein the fermenting is performed in the absence of added antibiotic.

20. The method of claim 12, wherein the fermentable carbohydrate-containing feedstock comprises flowable carbohydrate-containing feedstock derived from corn in an aqueous medium.

21. The method of claim 12, wherein the microorganism is a bacterium.

22. The method of claim 12, wherein the monochloramine is added to the fermentable carbohydrate-containing feedstock at a concentration of 0.1 ppm to 750 ppm, and the at least one peroxide compound is added to the fermentable carbohydrate-containing feedstock at a concentration of 0.1 ppm to 750 ppm.

23. The method of claim 12, wherein the monochloramine is present in the fermentable carbohydrate-containing feedstock at a concentration of 1 ppm to 450 ppm, and the at least one peroxide compound is present in the fermentable carbohydrate-containing feedstock at a concentration of 5 ppm to 450 ppm.

24. The method of claim 12, wherein the monochloramine and the at least one peroxide compound are added to the fermentable carbohydrate-containing feedstock in a ratio of 0.001:1 to 1:0.001.

25. The method of claim 12, wherein the peroxide compound is hydroperoxide, organic peroxide, inorganic peroxide, peroxy-releasing compound, or any combinations thereof.

26. The method of claim 12, wherein the peroxide compound is a hydroperoxide having the structure R-O-O-H, wherein R is a hydrogen or straight, branched and/or cyclic alkyl radical having 1 to 20 carbons atoms and can be optionally interrupted by one or more oxygen and/or carbonyl groups.

27. The method of claim 12, wherein the peroxide compound is an organic peroxide having the structure R′—O—O—R″, wherein R′ and R″ are independently straight, branched, and/or cyclic alkyl radical having 1 to 20 carbons atoms and can be optionally interrupted by one or more oxygen and/or carbonyl groups.

28. The method of claim 12, wherein the peroxide compound is an inorganic peroxide selected from alkali metal peroxide, alkaline earth metal peroxide, transition metal peroxide, or any combinations thereof.

29. The method of claim 12, wherein the peroxide compound is a peroxygen-releasing compound selected from alkali metal percarbonates, alkaline earth metal percarbonates, transition metal percarbonates, alkali metal perborates, alkaline earth metal perborates, transition metal perborates, or any combinations thereof.

30. The method of claim 12, wherein the pH of the fermentable carbohydrate-containing feedstock is from about 4 to about 7.

31. The method of claim 12, further comprising the steps of:
d) separating the stillage into a liquids-containing fraction and a solids-containing fraction;
e) optionally recycling at least portion of the liquids-containing fraction of d) into the fermenter vessel;
f) recovering the solids-containing fraction of d) with drying of at least a portion of the solids-containing fraction to produce evaporated vapors and distillers dried grains product free of antibiotics.

32. An aqueous solution comprising (a) monochloramine and (b) at least one peroxide compound, wherein components (a) and (b) are present in a synergistically microbicidally effective combined amount to control the growth of at least one microorganism.

33. The aqueous solution of claim 32, wherein the monochloramine is present in the aqueous solution at a concentration of 0.1 ppm to 750 ppm, and the at least one peroxide compound is present in the aqueous solution at a concentration of 0.1 ppm to 750 ppm.

34. The aqueous solution of claim 32 comprising the monochloramine is present in the aqueous solution at a concentration of 1 ppm to 450 ppm, and the at least one peroxide compound is present in the aqueous solution at a concentration of 5 ppm to 450 ppm.

35. The aqueous solution of claim 32, wherein the monochloramine and the at least one peroxide compound are added to the aqueous solution in a ratio of 0.001:1 to 1:0.001.

36. The aqueous solution of claim 32, wherein the peroxide compound is hydroperoxide, organic peroxide, inorganic peroxide, peroxy-releasing compound, or any combinations thereof.
